(19) United States
(12) Patent Application Publication
(10) Pub. No.: US 2014/0213917 A1
(43) Pub. Date: Jul. 31, 2014

(54) WATERPROOF HEART RATE MEASURING APPARATUS

(71) Applicant: Butterfleye SAL, Beirut (LB)

(72) Inventors: Hind Louis HOBEIKA, Yarze (LB); Benjamin CORRADO, Salem, OR (US); Donald CHEN, Salem, OR (US)

(21) Appl. No.: 14/242,257

(22) Filed: Apr. 1, 2014

Related U.S. Application Data
(63) Continuation-in-part of application No. 13/167,044, filed on Jun. 23, 2011.

(51) Int. Cl. A61B 5/024; A61B 5/00

(52) U.S. Cl. CPC A61B 5/02427; A61B 5/6803
USPC 600/500

(57) ABSTRACT

A biofeedback device and the light sensor used thereby are described herein that can be mounted on or integrated with eyewear such as swimming goggles. The biofeedback device may include a heart rate measurement apparatus comprising a reflected green light sensor, and first, second, and third green light emission elements. The biofeedback device may include a housing having a first portion and a second portion, which each of the first and second portions having a first side and a second side. At least a portion of the heart rate measurement apparatus may be disposed within the housing first portion and may be exposed through an opening in the second side of the housing first portion. The biofeedback device may also include an opening that allows the device to be removably engageable with at least a portion of the swimming goggles.

### CROSS-REFERENCE TO RELATED APPLICATION

This application is a Continuation-in-Part of, and claims priority to, U.S. Ser. No. 13/167,044, entitled HEART RATE WATERPROOF MEASURING APPARATUS, filed Jun. 23, 2011, the entirety of which is incorporated by reference. This application also claims the benefit of the following foreign application, which is incorporated herein by reference in its entirety: Lebanese Serial Patent No. 9099, filed Jul. 31, 2010.

### STATEMENT REGARDING FEDERALLY SPONSORED RESEARCH OR DEVELOPMENT

n/a

### FIELD OF THE INVENTION

The present invention relates to a waterproof heart rate measuring apparatus that can be mounted on or integrated with eyewear, including swimming goggles and sunglasses.

### BACKGROUND OF THE INVENTION

Heart rate monitoring is one of the most important tools for efficient cardiovascular training. As an indicator of not only the level of physical exertion but also the body's physiological adaptation to exercise, heart rate is a basis on which to gauge overall fitness. Additionally, monitoring heart rate is an easy way to make sure the body is not being dangerously overexerted. Many types of heart rate monitoring devices are known in the art, including devices that are worn around the wrist, on a finger, or around the torso, and those that use pressure, light, electrodes, and other methods to measure heart rate.

Heart rate is defined as the number of heart beats per unit of time, usually expressed as beats per minute (bpm), and can change as the body's need for oxygen changes in response to activity. The maximum heart rate, defined as the maximum safe heart rate for an individual, depends on factors such as age, sex, and fitness level of the individual. The most accurate way of measuring the maximum heart rate is through a cardiac stress test, in which the individual exercises while being monitored by an electrocardiograph (EKG). For general purposes, however, a formula is used to estimate Maximum Heart Rate:

HRₘₐₓ=220−age.

There is a direct relationship between heart rate and intensity of physical activity. Three different training zones are commonly used: weight loss, fitness, and maximum performance. If an individual wishes to lose weight, the individual should limit heart rate to 50% to 70% of the individual's maximum heart rate during exercise. To increase fitness, an individual should limit heart rate to 70% to 85% of maximum heart rate. An individual who wants to improve athletic performance should aim for a heart rate that is higher than 85% of the individual's maximum heart rate. In professional athletic training, an athlete may utilize all three heart rate zones for building cardiovascular health and endurance.

A number of heart rate sensors are known, including those that use sound, light, and/or pressure to measure the pulse. One type of sensor is an infrared plethysmograph. Such a sensor includes a photodiode that emits an infrared light and a phototransistor that receives the reflected infrared light. The superficial temporal artery, a major artery of the head that is located approximately 5 mm below the skin of the temple, provides ample blood volume for perfusion of blood around the temple. It is the smaller of the two branches of the external carotid artery, and its pulse is palpable superior to the zygomatic arch and anterior to and superior to the tragus. The pulse is calculated from the changes in volume of blood between the systole and diastole phases of the heart present in the tissues around the temple. In the diastole phase, the cavities of the heart are expanded and fill with blood, resulting in lower blood pressure and less blood volume in the capillaries. The heart contracts in the systole phase, resulting in higher blood pressure. The amount of blood in the tissues around the temple is directly related to its volume: more blood (higher volume) in the systole phase and less blood (lower volume) in the diastole phase. There is a slight increase in the light absorption by the tissues during the systolic phase, and less light is reflected back to the phototransistor of the sensor.

Infrared light may be used to measure heart rate in this fashion. Although using infrared light to monitor heart rate may be effective, blood volume between the temporal bone and the skin surface is relatively small, and the location of capillaries in the temporal area can vary between one person and another. Also, a person's skin and/or hair color may make it difficult for an infrared-light-based heart rate sensor to capture a good signal. Additionally, a person's movement during physical activity may create a substantial amount of noise that may affect the accuracy of a heart rate reading.

Athletes and participants in every sport can benefit from monitoring heart rate during training, including swimmers. Taking accurate and frequent heart rate measurements not only is useful in tracking changes in cardiovascular fitness over time and optimizing training, but also to prevent injury and exercise stress. If not correctly monitored, a swimmer can easily overtrain, which means that heart rate is so high that the swimmer is training in an anaerobic zone. Although anaerobic training can be a part of a balanced training program, an anaerobic workout can damage the muscle cell walls and result in decreased aerobic capacity for 24 to 96 hours. Consistently training in the anaerobic zone is counterproductive and can lead to injury and fatigue. The traditional method of measuring heart rate is to count the number of pulses over one minute. Heart rate measurements are of the greatest training value when measured during the physical activity, but it is difficult to accurately measure swimming heart rate using the wrist or neck pulse because of human error and the inconvenience of having to stop swimming long enough to measure heart rate. A heart rate monitoring device is preferable, but the device options are limited by the additional need for waterproofing and a practical means of communicating heart rate and other biofeedback data.

An effective heart rate monitor for swimmers must also be able to communicate current heart rate to the user in a way that does not disrupt training. Devices worn on the wrist, for example, are inconvenient because the user cannot see the display while swimming. Other devices may be able to display a number in the user's field of view, but the user must still concentrate enough to read the numbers. This may not be an easy task while the user is swimming quickly or is focused on stroke technique.

Also, some swimmers use certain training devices that do not interrupt swimming, such as pacing devices, timers, and lap counters. However, no device offers a combination of a heart rate monitor, pacing device, timer, lap counter, and other features such as pulse oximetry and calorie monitoring. Furthermore, no device displays heart rate to the user in a non-numeric method that the user can interpret easily while swimming.

It is therefore desirable to provide a waterproof heart rate monitoring device and system that is convenient to use during swimming and also is capable of measuring and recording other types of biofeedback and non-biofeedback data. It is also desirable to provide a device and system that include a method of wireless transmission so the measured biofeedback and non-biofeedback data could be sent from the device to a mobile phone or computer, or include an integrated memory chip that stores the data. Further, such a device and system should communicate heart rate to the user without requiring the user to divert attention away from training. Still further, the device and system should minimize or overcome noise interference and be usable by people with any of a variety of blood volume differences, skin colors, and hair colors.

### SUMMARY OF THE INVENTION

The present invention advantageously provides a biofeedback device, and the reflected light sensor used thereby, that can be mounted on or integrated with eyewear such as swimming goggles. In one embodiment, the heart rate measurement apparatus may include a first light emission element, a second light emission element, and a third light emission element, each of the first, second, and third light emission elements being configured to emit green light toward a target location, such as the temporal bone and/or tissues between the temporal bone and skin of a user's temple, and a light sensor configured to receive green light reflected from the target location through tissue between the temporal bone and the skin, the sensor being located between the first light emission element and the second light emission element, the first light emission element, sensor, second light emission element, and third light emission element being at least substantially collinear. The first light emission element may be located approximately 5 mm from the sensor in a first direction, the second light emission element may be located approximately 5 mm from the sensor in a second direction, and the third light emission element may be located approximately 10 mm from the sensor in the second direction. The first direction may be approximately 180° from the second direction. The apparatus may further include a first shield element between the first light emission element and the sensor, a second shield element between the sensor and the second light emission element, and at least one shield element between the second light emission element and the third light emission element. The first, second, and third light emission elements may be light-emitting diodes (LEDs). Further, the apparatus may be located within a housing. For example, the housing may include a first portion and a second portion, each of the first portion and the second portion having a first side and a second side. At least a portion of the housing first portion may lie in a first plane and at least a portion of the housing second portion may lie in a second plane, the first plane being at least substantially orthogonal to the second plane. Additionally, the second side of the housing first portion may include an opening sized to allow at least a portion of the heart rate measurement apparatus to extend therethrough. The first light emission element, the second emission element, and the third emission element may emit light through the opening in the second side of the housing first portion, and the sensor may detect light reflected from the reflection location through the opening in the second side of the housing first portion. The housing second portion may define an opening that extends from the second portion first side to the second portion second side, and the housing may further include a plurality of signal light emission elements disposed within the housing second portion. For example, the housing may be configured such that light emitted from the plurality of signal light emission elements may be visible through the second side of the housing second portion. The housing may be configured to be releasably engageable with an item of eyewear, such as swimming goggles having an eye cup, the eye cup defining an outer perimeter and including a lens having an anterior face. The housing second portion opening may be configured to be disposed about the outer perimeter of the eye cup, and the plurality of light emission elements may be configured to emit light onto the anterior face of the eye cup lens.

In one embodiment, a biofeedback device may include a housing defining a first portion and a second portion, each of the first portion and second portion defining a first side and a second side, the housing further defining a first opening in the second side of the housing first portion and a second opening that extends from the first side of the housing second portion to the second side of the housing second portion; a plurality of signal light emission elements disposed within the housing second portion and being configured to emit light through the housing second portion; and a heart rate measurement apparatus at least partially disposed within the housing, the heart rate measurement apparatus. The heart rate measurement apparatus may include a first light emission element, a second light emission element, and a third light emission element, each of the first, second, and third light emission elements being configured to emit light toward a target location; and a light sensor configured to receive light reflected from the target location, the sensor being located between the first light emission element and the second light emission element, the first light emission element, sensor, second light emission element, and third light emission element being at least substantially collinear and positioned within the first opening. The first light emission element, the second light emission element, and the third light emission element may be configured to emit green light and the sensor may be configured to detect green light reflected from the target location, such as the temporal bone and/or tissues between the temporal bone and skin. At least a portion of the housing first portion may lie in a first plane and at least a portion of the housing second portion may lie in a second plane, the first plane being substantially orthogonal to the second plane.

In one embodiment, a biofeedback system may include: a pair of swimming goggles including an eye cup defining an outer perimeter and including a lens defining an anterior face; a housing defining a first portion and a second portion, each of the first portion and second portion defining a first side and a second side, the housing further defining a first opening in the second side of the housing first portion and a second opening that extends from the first side of the housing second portion to the second side of the housing second portion; a plurality of signal light emission elements disposed within the housing second portion and being configured to emit light through the housing second portion; and a heart rate measurement apparatus at least partially disposed within the housing, the heart rate measurement apparatus including: a first light emission element, a second light emission element, and a third light emission element, each of the first, second, and third light emission elements being configured to emit green light toward a target location; and a light sensor configured to receive green light reflected from the target location, the sensor being located between the first light emission element and the second light emission element, the first light emission element, sensor, second light emission element, and third light emission element being at least substantially collinear and positioned within the first opening.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 shows a perspective view of a first embodiment of the waterproof biofeedback device;

FIG. 2A shows a perspective view of a first embodiment of a waterproof housing with a reflected infrared sensor contained therein;

FIG. 2B shows a sectional view of the reflected infrared sensor within the housing of FIG. 2A, the reflected infrared sensor being covered by a thin waterproof layer of material;

FIG. 3 shows a second embodiment of a waterproof heart rate measurement device;

FIG. 4 shows an alternate sectional view of the device of FIG. 3;

FIG. 5 shows a cross-sectional view of the reflected infrared sensor of the device and placement of the reflected infrared sensor on the skin above the temporal artery of the head;

FIG. 6A shows a cross-sectional view of the waterproof housing including an reflected infrared sensor and panel-type sensor adjustment mechanism;

FIG. 6B shows a sectional elevation view of the waterproof housing including the reflected infrared sensor and panel-type sensor adjustment mechanism;

FIG. 6C shows the spiral-type sensor adjustment mechanism;

FIG. 6D shows the waterproof housing containing the spiral-type sensor adjustment mechanism;

FIG. 7A shows a sectional view of the device having rope-type LEDs located on the circumference of the inner surface of a lens;

FIG. 7B shows a sectional view of the device having discrete LEDs located on the inner surface of a lens;

FIG. 8A shows a sectional view of the device having a signal element coupled to a eye cup track positionable element;

FIG. 8B shows a sectional view of the device having a signal element coupled to a suction cup positionable element;

FIG. 9 shows a schematic diagram of an exemplary function of the first and second embodiments of the biofeedback device;

FIG. 10 shows a first side view of the third embodiment of the waterproof biofeedback device that includes green light emitters and a green light receiver;

FIG. 11 shows a second side view of the third embodiment of the waterproof biofeedback device;

FIG. 12 shows a first perspective view of the third embodiment of the waterproof biofeedback device attached to a pair of swimming goggles;

FIG. 13 shows a second perspective view of the third embodiment of the waterproof biofeedback device attached to a pair of swimming goggles;

FIG. 14 shows a schematic view of the biofeedback device positioned next to a user's temple and emitting light toward the temporal bone;

FIG. 15A shows a close-up view of a first embodiment of the heart rate measurement apparatus of the third embodiment of the waterproof biofeedback device; and

FIG. 15B shows a close-up view of a second embodiment of the heart rate measurement apparatus of the third embodiment of the waterproof biofeedback device;

FIG. 16 shows an exemplary connection between the biofeedback device and a computer; and

FIG. 17 shows a flowchart of an exemplary operation of the third embodiments of the biofeedback device.

### DETAILED DESCRIPTION OF THE INVENTION

Monitoring heart rate is very important in an athletic training program, especially swimming. Although there are many available types of heart rate monitors, not all are waterproof and convenient for use while swimming. Furthermore, none of the available waterproof heart rate monitors combine a heart rate measurement apparatus with the measurement of time, calories burned, swim pace, swim duration, blood oxygen, distance traveled, and laps completed. The present invention advantageously provides a biofeedback device that can be waterproofed and mounted on or integrated with eyewear such as swimming goggles. Heart rate is then communicated to the user by one or more signal elements positioned within the user's field of vision (if visual), or otherwise communicated to the user (if auditory or tactile). The present invention also advantageously provides a reflected infrared sensor used within the device, the reflected infrared sensor having optimal geometry for detecting heart rate from subcutaneous blood vessels, such as the superficial temporal artery or other tissues between the temporal bone and the skin.

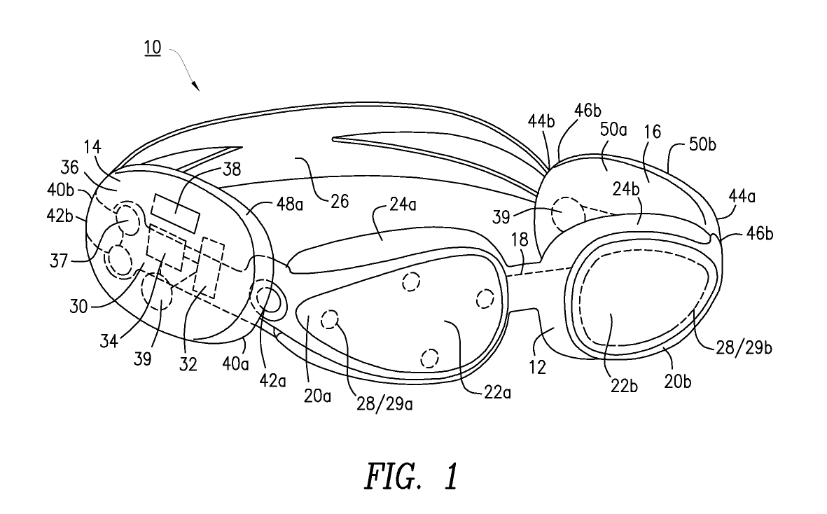

Referring now to FIG. 1, a first embodiment of the biofeedback device 10 is shown. The biofeedback device 10 may be usable with a pair of goggles 12, a first waterproof housing 14, a second waterproof housing 16, and one or more wires 18 for electrical communication between the first and second waterproof housing 14, 16. The goggles 12 may be a pair of traditional swimming goggles, or they may be any other type of protective eyewear. Goggles 12 suitable for use with the device 10 may comprise a first and second eye cup 20a, 20b, a first and second lens 22a, 22b, a first and second eye cup gasket 24a, 24b, and a head strap 26. The first and second eye cups 20a, 20b may be composed of any transparent or semi-transparent material, including polycarbonate, optical-grade plastic, or even glass. The first and second eye cup gaskets 24a, 24b may be composed of any material suitable for contact with the face, although silicone and foam are the most popular materials. However, the goggles 12 may not include the first and second eye cup gaskets 24a, 24b, as seen in Swedish goggles commonly used for competitive swimming. One or more signal elements 28, such as LEDs 29, either rope-type (29a) or discrete LEDs (29b), may be included within the interior of one or both of the eye cups 20a, 20b. The one or more signal elements 28 may comprise any type of visual, auditory, or tactile signaling system that can communicate heart rate, pace, or other measurements to the user, and may communicate such in a non-alphanumeric manner.

The one or more signal elements 28 shown in the figures is an LED system, and the LEDs 29 are discussed in more detail below. The head strap 26 may also be of any suitable material, although the most popular materials are silicone and rubber (which are resilient) and the typical bungee cord (a cord with a core composed of a plurality of elastic strands, covered in a woven polypropylene or cotton sheath). The head strap 26 may comprise a single strap, a split single strap, a double strap, or any variation that will securely hold the goggles 12 to the user's head.

Continuing to refer to FIG. 1, the first waterproof housing 14 may contain therein or have coupled thereto a heart rate measuring or measurement apparatus 30 comprising a reflected infrared sensor 32, a microcontroller 34, and a user interface 36. Although the term “heart rate measurement apparatus 30” is used herein for simplicity, it should be understood that the heart rate measurement apparatus 30 also may include circuitry that allows it to measure and record, in addition to heart rate, other biofeedback and non-biofeedback data such as calories burned and blood oxygen, and also data such as time, swim pace, swim duration, distance traveled, and laps completed. The user interface 36 may comprise one or more buttons 37 and one or more display screens 38, or it may additionally or alternatively comprise any other operable elements such as knobs, switches, touch screens, etc. The microcontroller 34 of the heart rate measurement apparatus 30 calculates the heart rate. The reflected infrared sensor 32 transmits signals of voltage per unit of time to the microcontroller 34, which may comprise one or more filters that filter all noise coming from electromagnetic interference and from ambient or environmental light and one or more amplifiers that amplify the remaining signal. The microcontroller 34 may then digitally filter the signal to extract the alternating current (AC) component of the signal, and then evaluate the time (T) between two pulses. The microcontroller 34 follows a formula to calculate the heart rate:

Heart Rate=60/T

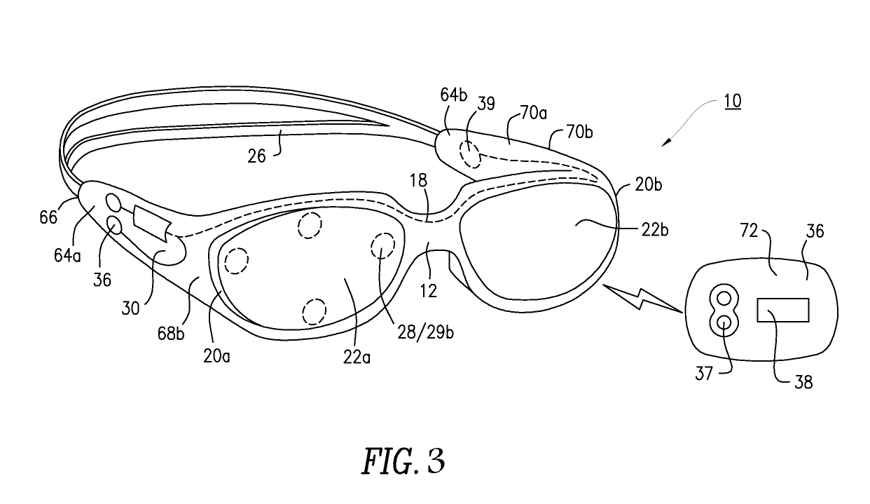

To obtain an accurate measurement over time, every five heart rate measurements may be averaged by the microcontroller 34 to obtain a moving average heart rate. A comparator may compare between the heart rate measurement and the target heart rate (calculated by the microcontroller 34 based on data entered in the user interface 36). Further, the microcontroller 34 may include a wireless communication interface adapted to be in wireless communication with a wireless data network, enabling transmission of recorded data to a computer, mobile phone, or other wireless device, or an integrated memory chip. The user interface 36 may also be in wireless communication with a wireless remote keyboard and display device, such as a dedicated device, mobile phone, PDA, or any other suitable device that is operable on wireless networks such as BLUETOOTH® or Wi-Fi. Additionally, the user interface 36 may be disposed within the first waterproof housing 14, or it may be housed in a remote device 72 in wireless communication with the microcontroller 34 (shown in FIG. 3). For simplicity, the term “microcontroller” as used herein may include the one or more filters, one or more amplifiers, comparator, wireless interface, and any other circuitry used to receive signals from the reflected infrared sensor 32 and perform calculations to produce final measurements and communicate said measurements to the user through a display element 28.

Continuing to refer to FIG. 1, the second waterproof housing 16 may contain therein a power source 39 that may be rechargeable or single use, for example a small battery such as a hearing aid or watch battery (button cell). The first and second waterproof housings 14, 16 may be composed of any rigid or semi-rigid, lightweight, waterproof material, such as acrylic, to prevent water and humidity from entering the housing and coming in contact with the electronic elements, to protect the unit against shock damage (such as when the biofeedback device is dropped), and to increase stability to ensure accurate heart rate measurements. The housing shape may be oval or rounded to increase hydrodynamic efficiency, and the first and second waterproof housings 14, 16 each may include a mechanism (such as with a latch or screws) by which the user may open the waterproof housing to change the power source 39, adjust the reflected infrared sensor 32, or make repairs. All measurements taken by the reflected infrared sensor 32 rely on the accurate emission, reflection, and reabsorption of infrared light. Therefore, it is important to exclude as much ambient or environmental light as possible. To achieve this, the housing may further be coated with a layer of opaque material to block any interference by ambient or environmental light.

One or more wires 18 may put the first and second waterproof housings 14, 16 in electrical communication with each other and with the one or more signal elements 28 (if wireless communication is not used). These wires 18 may be disposed within a chamber defined by the frame of the goggles 12 that extends between the first and second waterproof housings 14, 16 and the one or more signal elements 28. The wires 18 and may be rigid enough to be easily fed through the chamber so the waterproof housings 14, 16 and one or more signal elements 28 may be completely removed from the goggles 12. Furthermore, the wires 18 may be coupled to a connection means on both ends so the wires 18 can be readily connected and disconnected from the waterproof housings 14, 16 and one or more signal elements 28. Alternatively, the housings 14, 16 may each be completely removable from a piece of eyewear, such as the goggles 12, and the one or more wires 18 may be disposed on the outside of the housing unit 13 and the eyewear.

Continuing to refer to FIG. 1, the first and second waterproof housings 14, 16 may be held securely against the skin of the user by the head strap 26, and the user may position the first and second housings for comfort and accuracy. The first waterproof housing 14 may have a first end 40a including a first strap attachment means 42a and a second end 40b including a second strap attachment means 42b, and the second waterproof housing 16 may have a first end 44a including a first strap attachment means 46a and a second end 44b including a second strap attachment means 46b, each strap attachment means 46a, 46b defining an opening through which the head strap 26 of the goggles 12 may pass. The first and second waterproof housings 14, 16 also may each have a first surface 48a, 50a and second surface 48b, 50b, the first surface 48a, 50a being in contact with the user's head and the second surface 48b, 50b being in contact with the head strap 26. The second surface 48b of the first waterproof housing 14 may include the user interface 36.

Continuing to refer to FIG. 1, it is understood that the heart rate measurement apparatus 30 (user interface 36, microcontroller 34, and reflected infrared sensor 32), power source 39, wires 18, and any other necessary components may be housed within a single waterproof housing. The power source 39 is shown in the first waterproof housing 14 in FIG. 1 because it may optionally be included in the first waterproof housing 14, with the second waterproof housing 16 being removed from the biofeedback device 10. All other elements of the biofeedback device 10 are as described for the biofeedback device 10 shown in FIG. 1.

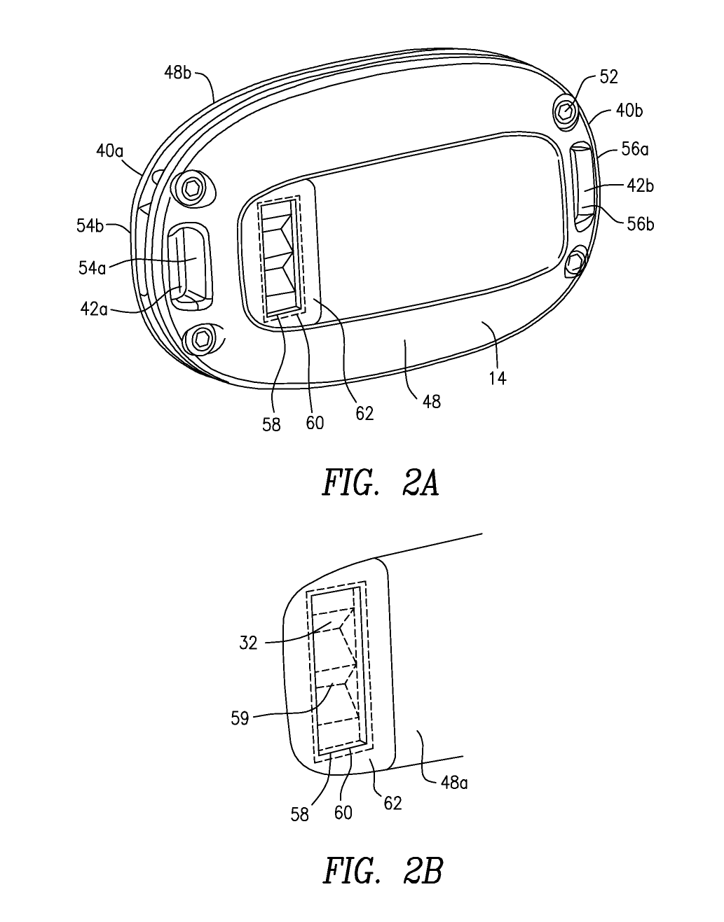

Now referring to FIGS. 2A and 2B, the first surface 48a of the first waterproof housing 14 is shown. One or more screws 52 may be used to seal the housing 14 against water and other environmental contaminants. As is also shown in FIG. 1, the first waterproof housing 14 may have a first end 40a and second end 40b, the first end 40a including a first strap attachment means 42a and the second end 40b including a second strap attachment means 42b. The first and second strap attachment means 42a, 42b each define an opening that may be wide enough to accommodate a typical head strap 26 (for example, the width may be approximately 0.2 cm to 1.0 cm), and may be tall enough to accommodate a typical head strap (for example, the height may be 0.5 cm to 2.0 cm). Each strap attachment means 42a, 42b opening may have an entry 54a, 56a on or adjacent the first surface 48a of the first waterproof housing 14 and an exit 54b, 56b on or adjacent the second surface 48b of the first waterproof housing 14 through which the head strap 26 may pass. For example, to attach the first waterproof housing 14 to the goggles 12 and ensure contact with the user's skin, the head strap 26 may be fed into the entry 54a of the first strap attachment means 42a, then out the exit 54b of the first strap attachment means 42a. The head strap 26 may then be in contact with the second surface 48b of the first waterproof housing 14, passing from the first end 40a to the second end 40b. Finally, the head strap 26 may be fed into the entry 56a and out the exit 56b of the second strap attachment mechanism 42b. The first and second waterproof housings 14, 16 may each be positioned at any location on the strap 26 relative to the user, such as in the back of the user's head or on either side of and immediately adjacent to the eye cups 20a, 20b. Although not shown in FIG. 2A or 2B, it is understood that the second waterproof housing 16, also having a first and second strap attachment means 46a, 46b, may be attached to the goggles 12 in a similar manner.

Continuing to refer to FIGS. 2A and 2B, the first waterproof housing 14 may have a sensor opening 58 through which the reflected infrared sensor 32 is exposed to the skin of the user. The dimensions of the sensor opening 58 may be the same as the dimensions of the area of the sensor 32 that is exposed to the skin. The reflected infrared sensor 32 may be entirely disposed within the first waterproof housing 14, whereas the reflected infrared sensor 32 may be substantially coterminous with the sensor opening 58 in the first housing 14. Because the reflected infrared sensor 32 may be composed of a nonconductive waterproof material, such as Teflon, the sensor opening 58 and at least part of the reflected infrared sensor 32 may be exposed to the water and in direct contact with the skin (as shown in FIG. 2A), or the reflected infrared sensor 32 may be covered by a thin layer of insulation material that allows the transmission of infrared light therethrough, such as silicone 59 (as shown in FIG. 2b). A gasket 60 (such as a typical rubber O-ring) may be included inside the first waterproof housing 14, between the reflected infrared sensor 32 base and the first surface 48a of the first waterproof housing 14, to prevent the entry of water into the housing. Additionally, a portion of the first surface 48a surrounding the outer perimeter of the sensor opening 58 may be covered in a waterproof, opaque material with a relatively high coefficient of friction on skin (approximately 0.3 to 1.0μ), such as rubber. This outer perimeter may help ensure maximum contact and stability between the reflected infrared sensor 32 and the user's skin, thereby increasing the accuracy of the reflected infrared sensor 32's measurements. For simplicity, the area of the first surface 48a of the first waterproof housing 14 is referred to herein as the rubber pad 62, even though it may be composed of a different material.

Referring now to FIG. 3, a second embodiment of the biofeedback device 10 is shown. In this embodiment, the heart rate measurement apparatus 30, power source 39, and one or more wires 18 are entirely disposed within the frame of the goggles 12. The frame of the goggles 12 may be impervious to water and other environmental contaminants similar to the first and second waterproof housings 14, 16 shown in FIGS. 1, 2A, and 2B and discussed above. The frame of the goggles 12 may include a first arm 64a and a second arm 64b, each arm having a strap attachment means 66 at the terminus. The strap attachment means 66 may comprise a metal or plastic cap and loop through which the head strap 26 may be secured; however, any type of strap attachment means may be used that will securely couple the head strap 26 and goggles 12. The first arm 64a and the second arm 64b each have a first surface 68a, 70a and a second surface 68b, 70b, each first surface 68a, 70a being in contact with the user's head. The heart rate measurement apparatus 30 and the power source 39 may be in electrical communication with each other via one or more wires 18 disposed within a channel defined by the frame of the goggles 12 (if wireless communication is not used). The heart rate measurement apparatus 30 may be entirely disposed within the first arm 64a of the goggles 12, except that the reflected infrared sensor 32 may be exposed to the water or user's skin through an opening 61 on the first surface 68a of the first arm 64a. Similarly, the one or more buttons 37, display screens 38, or other user control features of the user interface 36 are located on the second surface 68b of the first arm 64a, where they are accessible to the user. The power source 39 may be entirely disposed within the second arm 64b of the goggles 12. It is understood, however, that the user interface 36 and heart rate measurement apparatus 30 may be alternatively disposed within the second arm 64b, and the power source 39 may be disposed within the first arm 64a.

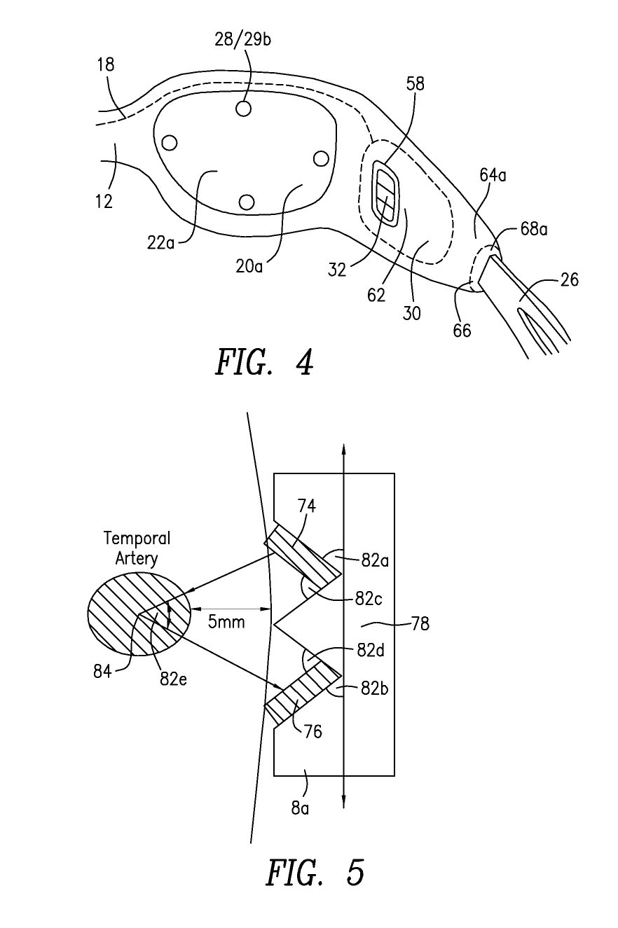

Continuing to refer to FIG. 3, the user input may alternatively be located on a remote device 72 in wireless communication with the microcontroller 34 of the heart rate measurement apparatus 30. Thus, the heart rate measurement apparatus 30 in this alternative embodiment may comprise the reflected infrared sensor 32 and microcontroller 34, but not the user interface 36. Including the user interface 36 in a separate from the goggles 12 may allow for a more streamlined design of the biofeedback device 10, as seen in FIG. 4. The remote device 72 may include one or more buttons 37, display screens 38, and other user control elements. The user would enter into the remote device 72 age, weight, target heart rate, workout time, and other data useful in calculating calories burned, workout time, stroke pacing, and other parameters. Additionally, the user interface 36, either disposed within the biofeedback device 10 or remote device 72, could be used for selecting or creating a desired training program. The remote device 72 would wirelessly transmit this data (such as by WiFi, infrared, or BLUETOOTH® signal) to the microcontroller 34 of the heart rate measurement apparatus 30, which would, in turn, operate the one or more signal elements 28 accordingly (e.g., color of light and/or pace of blinking of LEDs 29). The remote device 72 may include therein a power source 39 that may be rechargeable or single use, for example a small battery such as a hearing aid or watch battery (button cell), and may be waterproof like the first and second waterproof housings 14, 16 shown in FIGS. 1, 2A, and 2B, and discussed above. It should be understood that the remote device configuration may be used with either the integrated or non-integrated heart rate measurement apparatus design (for example, either the biofeedback device 10 of FIG. 1 or the biofeedback device of FIG. 3).

Referring now to FIG. 4, an inside view of the first arm 64a of the goggles 12 is shown. The first surface 68a of the first arm 64a is shown, which includes an opening 61 through which the reflected infrared sensor 32 may be exposed to the user's skin. The reflected infrared sensor 32 may be composed of waterproof materials and therefore may be exposed to the water and in direct contact with the user's skin; however, the reflected infrared sensor 32 may alternatively be covered by a thin layer 59 of insulation material that allows the transmission of infrared light therethrough without distorting the infrared signal (as shown in FIG. 2b).

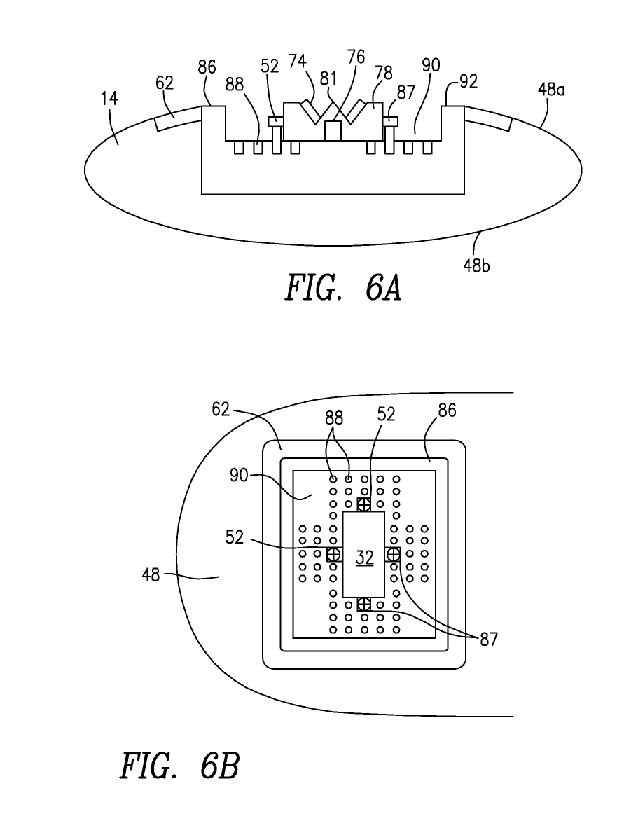

Referring now to FIG. 5, a cross section of the reflected infrared sensor 32 is shown, which may or may not be drawn to scale. The reflected infrared sensor 32 may comprise an infrared emitter 74 (photodiode), an infrared receiver 76 (phototransistor), and sensor base 78 having a first end 80a and a second end 80b, the sensor base 78 defining a shield element 81 to prevent the possible interference between the emitted and received infrared signals (i.e. to prevent the infrared light emitted from the infrared emitter 74 from directly entering the infrared receiver 76 without first being reflected from the target reflection point 84). The shield element 81 may be any size and shape sufficient to prevent the infrared signal interference, such as triangular shape. The reflected infrared sensor 32 may be composed of a nonconductive material, such as Teflon, to prevent interference with the current in the infrared emitter 74 and infrared receiver 76. Additionally, the material may be opaque and non-reflective in order to block any light that can interfere with the infrared light emitted by the infrared emitter 74 and/or distort the signal received by the infrared receiver 76. For simplicity, the term “reflected infrared sensor” used herein includes the infrared emitter 74, infrared receiver 76, and shield element 81. The reflected infrared sensor 32 may be placed in contact with the user's skin proximate a target area. For example, proximate the temporal artery (which may be located approximately 5 mm beneath the skin of the temple) or proximate other tissues between the temporal bone and the skin of the temple. It will be understood that the term “target reflection point” may be used to refer to any reflection point within a user's temple that emits sufficient light to the infrared receiver 76 for the infrared receiver 76 to detect a good heart rate or other biofeedback signal, and may not be in a specific location but rather a location that varies between users.

Continuing to refer to FIG. 5, the cross-sectional view of the reflected infrared sensor 32 may resemble the letter “W.” The infrared emitter 74 may be positioned at a first angle 82a measured in relation to an axis running from the first end 80a of the sensor base 78 to the second end 80b of the sensor base 78, and the infrared receiver 76 may be positioned at a second angle 82b measured in relation to said axis. Further, the infrared emitter 74 and the shield element 81 may define a third angle 82c, and the shield element 81 and the infrared receiver 76 may define a fourth angle 82d. The reflected infrared sensor 32 configuration may be determined for any target reflection point 84. For example, the angle between the infrared emitter 74 and the shield element 81 may be set at 45 degrees. Next, a point 5 mm from the outer edge of the infrared emitter 74 may be used as the reflection point because the temporal artery is located an average of 5 mm beneath the skin of the temple (as shown in FIG. 5). Then, the distance between the infrared emitter 74 and infrared receiver 76 may be adjusted until an oscilloscope measurement of the infrared signal is of the highest amplitude, which means the location of the infrared receiver 76 would ensure optimal receipt of the infrared light. The degree of emission (the fifth angle 82e) of the infrared light from the infrared emitter 74 may also be determined, based on the relative positions of the infrared emitter 74, infrared receiver 76, and the target reflection point 84.

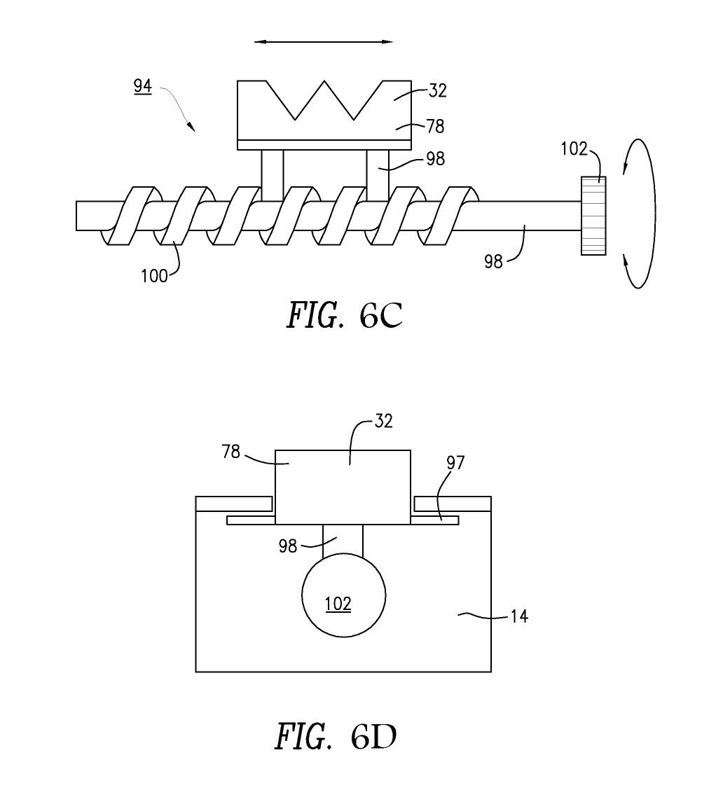

Referring now to FIGS. 6A, 6B, 6C, and 6D, the reflected infrared sensor 32 may be adjusted by the user horizontally (along an x-axis), vertically (along a y-axis), or a combination of horizontally and vertically to a distance of, for example, 1 cm. Since there are minimal variations between the location of the temporal artery between one person and another, the reflected infrared sensor 32 may be mounted within the waterproof housing (either in, for example, the first waterproof housing 14 or the first arm 64a of the goggles 12) in such a way that allows for the positioning of the reflected infrared sensor 32 by tightening or loosening one or more screws 52, while still preventing the entry of water into the waterproof housing. If the reflected infrared sensor 32 does not detect the user's heart rate, the one or more signal elements 28 will not broadcast a visual, auditory, or tactile heart rate signal to the user, but may instead emit a blinking red light. In this case, the user may adjust the reflected infrared sensor 32 until heart rate is detected. Unlike other heart rate measurement devices, the reflected infrared sensor 32 may not be easily repositioned by repositioning the entire device 10, because the goggles 12 must be fitted over the eyes of the user and thus may not be able to accommodate movement of a fixed sensor. Exemplary methods of adjusting the reflected infrared sensor are shown in FIGS. 6A, 6B, 6C, and 6D.

FIG. 6A shows a cross-sectional view of the first waterproof housing 14 with a panel-type sensor adjustment mechanism 86. The reflected infrared sensor 32, or a plurality of reflected infrared sensors 32, may be coupled to the panel-type sensor adjustment mechanism 86 by one or more screws 52 that may be screwed into any of a plurality of screw holes 88 located on the surface 90 of the panel-type sensor adjustment mechanism 86. The screw holes 88 may terminate at least partially through, but do not continue all the way through, the panel-type sensor adjustment mechanism 86, which prevents water from entering the first waterproof housing 14. The panel-type sensor adjustment mechanism 86 may be coupled to the first waterproof housing 14 such that only the outer rim 92 of the panel-type sensor adjustment mechanism 86 may be flush with the first surface 48a of the first waterproof housing 14, with the surface 90 of the panel-type sensor adjustment mechanism 86 being recessed. Similarly, the portion of the reflected infrared sensor 32 that is in contact with the skin may be substantially coplanar with the first surface 48a of the first waterproof housing 14.

Referring now to FIG. 6B, the panel-type sensor adjustment mechanism 86 may be adjusted horizontally (along an x-axis), vertically (along a y-axis), or a combination of horizontally and vertically by unscrewing the one or more screws 52 from any of a plurality of screw holes 88, moving the reflected infrared sensor 32 along the surface 90 of the panel-type sensor adjustment mechanism 86, and replacing the one or more screws 52 into the corresponding one or more screw holes 88. The sensor base 78 may also have one or more flanges 87 having one or more screw holes 88 that align with the one or more screw holes 88 on the surface 90 of the panel-type sensor adjustment mechanism 86. The entire surface 90 and outer rim 92 of the panel-type sensor adjustment mechanism 86 are waterproof and may be exposed to water.

Alternative or additional to the method of adjusting the reflected infrared sensor 32 shown in FIGS. 6A and 6B, a spiral-type sensor adjustment mechanism 94 may be included (as shown in FIGS. 6C and 6D). In the spiral-type sensor adjustment mechanism 94, reflected infrared sensor 32 may or may not be coupled to a surface having a plurality of screw holes 88. Instead, the infrared sensor 32 may be coupled to an adjustment plate 97 disposed within or coupled to the first waterproof housing 14. As shown in FIG. 6C, the sensor base 78 may include one or more feet 98 that may be in contact with a shaft 98 having a spiraled threading 100 (for example, a screw). As shown in FIG. 6D, the one or more feet 96, the shaft 98, and the spiraled threading 100 may be entirely disposed within the first waterproof housing 14. Coupled to one end of the shaft 98 may be a knob 102, which is not disposed within the first waterproof housing 14, but is instead accessible to the user. When the user turns the knob either clockwise or counterclockwise, the spiraled threading 100 engages the feet 96 to move the reflected infrared sensor 32 along either the x-axis or the y-axis (for example, to a distance of 1 cm from the center point in either direction), depending on the axis on which the spiral-type sensor adjustment mechanism 94 is disposed. It is understood that the sensor adjustment mechanisms 86, 94 of FIGS. 6A-6D could be similarly disposed within other waterproof housings, for example, the first arm 64a of the goggles 12.

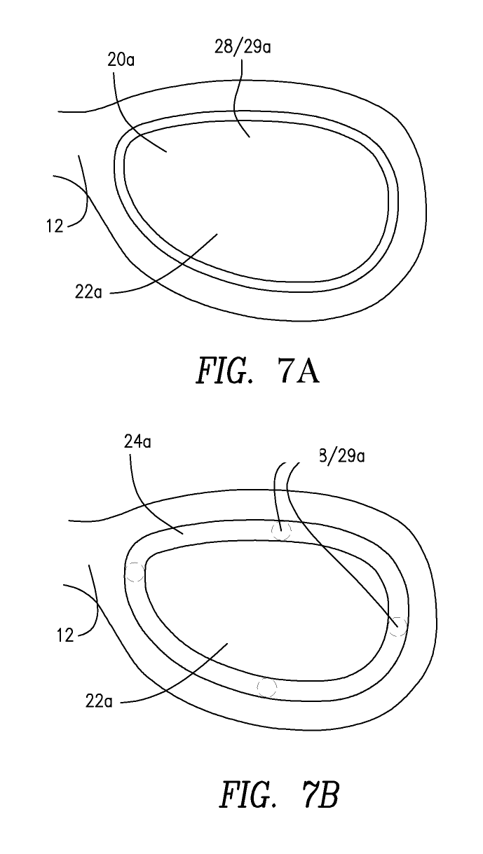

Referring now to FIGS. 7A and 7B, the one or more signal elements 28 are shown. FIG. 6A shows a continuous rope of clear tubing with multiple LEDs 29 therein 29a, and FIG. 7B shows discrete LEDs 29b. The clear tubing may contain one or more LEDs 29, and is referred to herein as a “rope-type LED light” 29a. Each eye cup 20a, 20b includes a lens 22a, 22b, which is the surface of the eye cup that is disposed directly in front of the user's eye. The rope-type LED light 29a may be at least partially disposed about the inner circumference of at least one of the first and second eye cups 20a, 20b either adjacent to or on the lens 22a, 22b. Included in the first eye cup 20a is a first lens 22a, and included in the second eye cup 20b is a second lens 22b.

The rope-type LED light 29a may be entirely disposed about a circumference of at least one of the first and second lenses 22a, 22b. For example, FIG. 7A shows the rope-type LED light 29a disposed about the entire inner circumference of the first eye cup 20a. Alternatively, the rope-type LED light 29a may be disposed within or underneath at least one of the first and second eye cup gaskets 24a, 24b, at least partially disposed about the inner circumference of the eye cup 20a, 20b where the eye cup 20a, 20b is coupled to the eye cup gasket 24a, 24b. Depending on the placement of the rope-type LED light 29a, the user may either perceive a direct light or an indirect light. When the rope-type LED light 29a is disposed within at least one of the first and second eye cup gaskets 24a, 24b, the light may be a diffuse light that is reflected from the inside of the eye cup 20a, 20b and may give the effect of illuminating the entire eye cup with color. No matter what the placement of the rope-type LED light 29a, the user should be able to perceive the color and/or blinking of the light without undue effort.

Continuing to refer to FIG. 7B, one or more discrete LEDs 29b are shown. The discrete LEDs 29b may be located at any position about the inner circumference of at least one of the first and second eye cups 20a, 20b, either adjacent to or on the first and/or second lens 22a, 22b. Any number of discrete LEDs 29b may be used. The discrete LEDs 29b may be equidistant from one another, or they may be grouped together at any point in the first and/or eye cup 20a, 20b. Alternatively, the discrete LEDs 29b may be disposed within or underneath at least one of the first and second eye cup gaskets 24a, 24b (as shown in FIG. 7B). Depending on the placement of the discrete LEDs 29b, the user may either perceive a direct light or an indirect light. When the discrete LEDs 29b are disposed within at least one of the first and second eye cup gaskets 24a, 24b, the light may be a diffuse light that is reflected from the inside of the eye cup 20a, 20b and may give the effect of illuminating the entire eye cup with color. No matter what the placement of the discrete LEDs 29b, the user should be able to perceive the color and/or blinking of the light without undue effort.

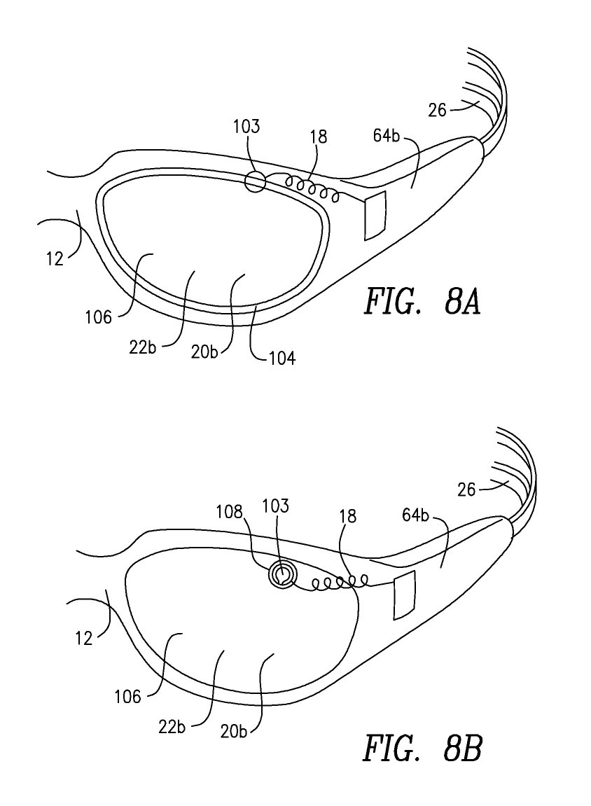

Referring now to FIGS. 8A and 8B, the one or more signal elements 28 may alternatively be coupled to or housed in a positionable element 103 that the user may place in any desired position on the biofeedback device 10. Such a housing may have such attachment means as a clip, adhesive junction, suction cup, malleable arm coupled to the goggles, or any other suitable means. For example, FIG. 8A shows the goggles 12 having an eye cup track 104 that may be disposed at least partially about the circumference of the outer surface 106 of one or both eye cups 20a, 20b. The one or more signal elements 28 may be removably coupled to the eye cup track 104, such as by a clip. FIG. 8B shows the one or more signal elements 28 coupled to a suction cup 108 that may be removably attached to the outer surface 106 of one or both eye cups 20a, 20b. Regardless of the type of positionable element 103 used, the positionable element 103 may be in electrical communication with the power source 39 and microcontroller 34 via one or more flexible wires 18 that may be at least partially disposed on the outside of the goggles 12 (not within a waterproof housing).

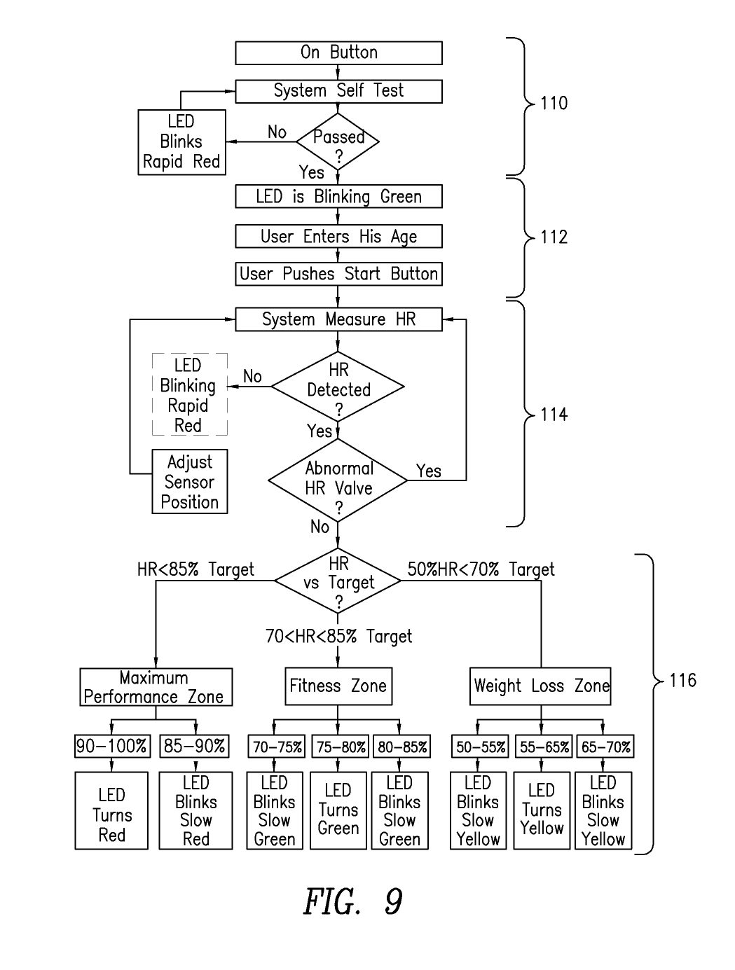

Referring now to FIG. 9, an exemplary communication scheme of the one or more signal elements 28 is shown. In FIG. 9, a visual signal element is contemplated, specifically, an LED display. Three colors of LEDs 29 may be used to represent the three training zones (weight loss, fitness, and maximum performance). It is understood that more colors may be used, depending on the number of training zones to be represented. Additionally, the LEDs 29 may emit a steady light only, or may emit a steady light or a blinking light to represent upper and lower ends of the represented training zones. The LEDs 29 may emit a blinking red light if the reflected infrared sensor 32 does not detect a heart rate. The presence of a blinking light will communicate to the user that the unit has sufficient power, but that the sensor is not in the optimal location for detecting heart rate. Further, the color of the light and its status (blinking or steady) easily communicate heart rate to the user without requiring the user to read small numbers or pause swimming to look at a watch or similar device.

FIG. 9 shows an example of this system: after a boot up sequence 110, the user may enter data into the user interface 36 (such as age, weight, or desired workout program), the process referred to as “user data entry” 112. The heart rate measurement apparatus 30 may then detect and measure the user's heart rate, and the user may manually adjust the position of the reflected infrared sensor 32 if no heart rate is detected. This process is referred to as “heart rate detection and adjustment” 114. After heart rate detection and measurement 114, heart rate measurement apparatus 30 may then compare the user's heart rate to the user's target heart rate and communicate the result to the one or more signal elements 28, a processed referred to as “comparison and display” 116.

FIG. 9 also shows an exemplary comparison and display 116 process, in which the weight loss zone is typically a heart rate of 50% to 70% of the maximum heart rate, and may be represented by one or more green LEDs 29. The green LEDs 29 may blink slowly in the 50% to 55% range (lower end of the zone), may glow steadily in the 55% to 65% range (middle of the zone), and may blink quickly in the 65% to 75% range (upper end of the zone). The fitness zone is typically a heart rate of 70% to 85% of the maximum heart rate, and may be represented by one or more yellow LEDs 29. The yellow LEDs 29 may blink slowly in the 70% to 75% range (lower end of the zone), may glow steadily in the 75% to 80% range (middle of the zone), and may blink quickly in the 80% to 85% range (upper end of the zone). The maximum performance zone is typically a heart rate of 85% of the maximum heart rate and above, and may be represented by one or more red LEDs 29. The red LEDs 29 may glow steadily in the 85% to 90% range (lower end of the zone), and may blink slowly at heart rates above 90% of the maximum heart rate (middle and upper end of the zone). Depending on the LEDs 29 used, any number of color options may be available for a single LED bulb (such as when multi-color LEDs 29 are used, or when the signal display element comprises multiple LEDs 29 of various colors). The user interface 36 may include a means by which the user may adjust the LED display correlated to heart rate. For example, the user may prefer blue LEDs 29 for the weight loss zone, red LEDs 29 for the fitness zone, and green LEDs 29 for the maximum performance zone. Additionally, the user may also use the user interface 36 to specify a steady LED glow without blinking, or may desire to set the speed of the blinking to match a target swim stroke pace.

It should be understood that the microcontroller 34 may measure and record other types of biofeedback data in addition to heart rate, and may also be able to measure non-biofeedback data. For example, the microcontroller 34 of the biofeedback device 10 may additionally comprise circuitry for performing the functions of a chronometer, timer, lap counter, distance measurement device, calorie counter, blood oximeter, and wireless transceiver (such as a BLUETOOTH® device).

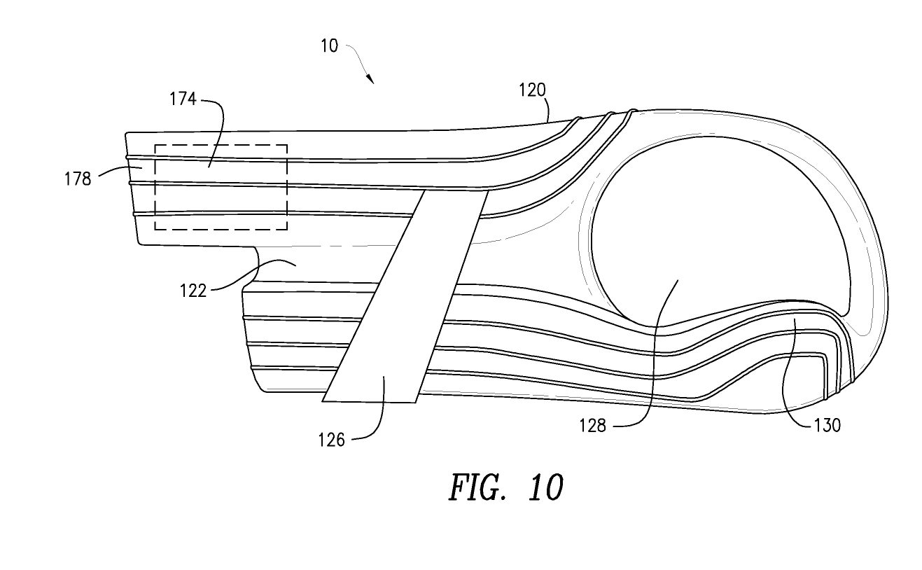

Referring now to FIGS. 10-17, a third embodiment of a waterproof biofeedback device 10 is shown. The device 10 shown in FIGS. 10-17 may be used in association with, and may be completely detachable from, a piece of eyewear such as swimming goggles 12. The device 10 may include a housing unit 120 generally having a first side 122 and a second side 124. The housing unit 120 may have a streamlined, hydrodynamic shape that is configured to minimize drag in water. For example, the housing unit 120 may have rounded edges, smoothly curved transitions between various portions of the housing unit, may be composed of a low-friction material, and/or may include fluid channels for directing fluid over the surface of the device 10. These features may be particularly important if the user is using the device 10 with a pair of swimming goggles for swim training. In use, the second side 124 of the device 10 may be an inner surface that is in contact with the user's skin and the first side 122 of the device 10 may be an outer surface that is opposite the second side 124. The first side 122 may include a strap attachment element 126, such as a clip, loop, ring, or hook. The strap attachment element 126 may be composed of a lightweight and waterproof material, such as aluminum or plastic. The device 10 in FIGS. 10-17 is shown as having a swimming goggle strap attachment clip 126, although it is contemplated that the strap attachment element could be adapted for use with other types of eyewear. For example, the attachment element 126 could be adapted to receive an arm of a pair of sunglasses.

The housing unit 120 may define an opening 128 sized to be placed in front of an eye cup 20 of a pair of goggles 12 or a lens of an item of eyewear without obscuring the user's view. Further, the housing unit 120 may define an LED housing portion 130 proximate or defining the lower margin of the opening 128. The LED housing portion 130 may be somewhat protuberant or raised from the rest of the first surface 122 or may be flush or substantially flush with the first surface 122. Further, the device 10 may contain one or more light emission elements, such as one or more light-emitting diodes (LEDs) 132, generally within the housing unit 120, and particularly within the LED housing portion 130. The housing 130 may be composed of a flexible material, such as a thermoplastic elastomer (TPE) or a TPE blend (for example, TPE and silicone), and may include a rigid plastic core supporting the circuitry.

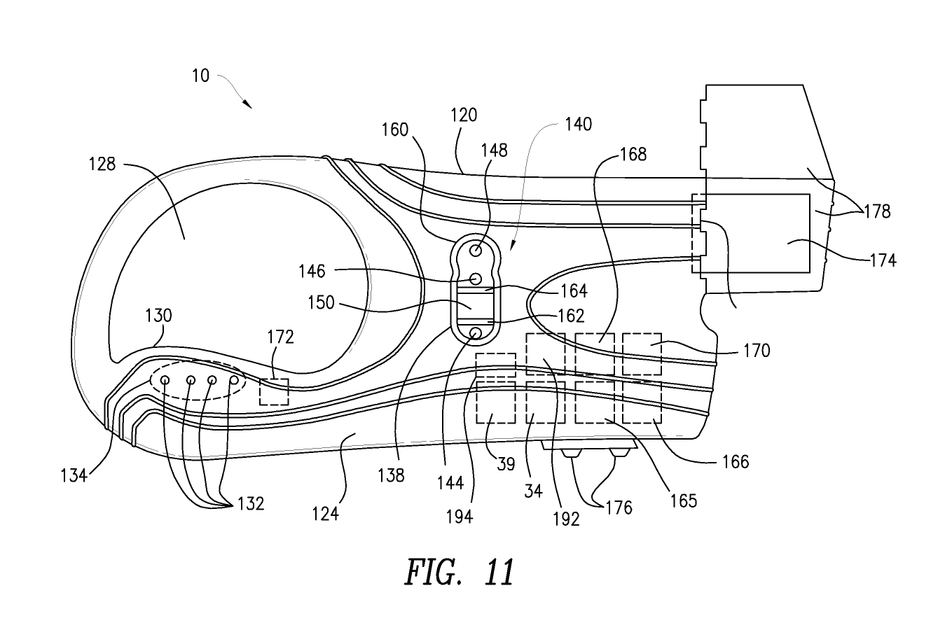

The one or more LED lights 132 may be visible through the housing unit 120. For example, the material of the LED housing portion 130 may be colorless or may be thin enough for the light emitted by the one or more LED lights 132 to be visible to the user through the housing unit 120. Alternatively, the one or more LED lights 132 may extend through one or more watertight openings in the LED housing portion 130, or the LED housing portion 130 may include a waterproof opening or window for each of one or more clusters of LEDs. The one or more LEDs 132 within the LED housing portion 130 may together comprise a display area 134 (shown generally with dashed lines in FIG. 11) that may communicate heart rate and other biofeedback data to the user during activity. However, the display area 134 may not be directly visible to the user. Instead, light emitted by the one or more LEDs 132 in the display area 134 may be reflected by a portion of an eye cup 20 that is in the user's line of vision, and the user may therefore see a general area of colorized light in the eye cup 20. Further, the one or more LEDs 132 may be in electrical communication with a power source 39 via a flex circuit rather than a fiber optic array. Although four LEDs are shown, for example, in FIG. 11, any number of LEDs may be used. Additionally, the LEDs 132 in any embodiment may have any of a variety of and shapes, including square, rectangular, or round. Further, the LED housing portion 130 may additionally or alternatively include a screen or display that could show data in a manner visible to the user. As discussed in more detail below, the device 10 may be used in connection with online, downloadable, or installable, and/or mobile application software, and the colors of LEDs used and other characteristics of the display area 134 may be set according to the user's preferences using this software.

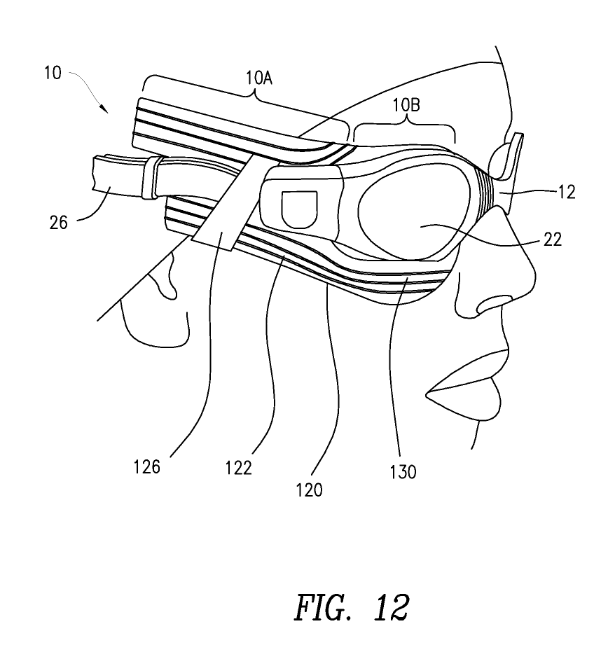

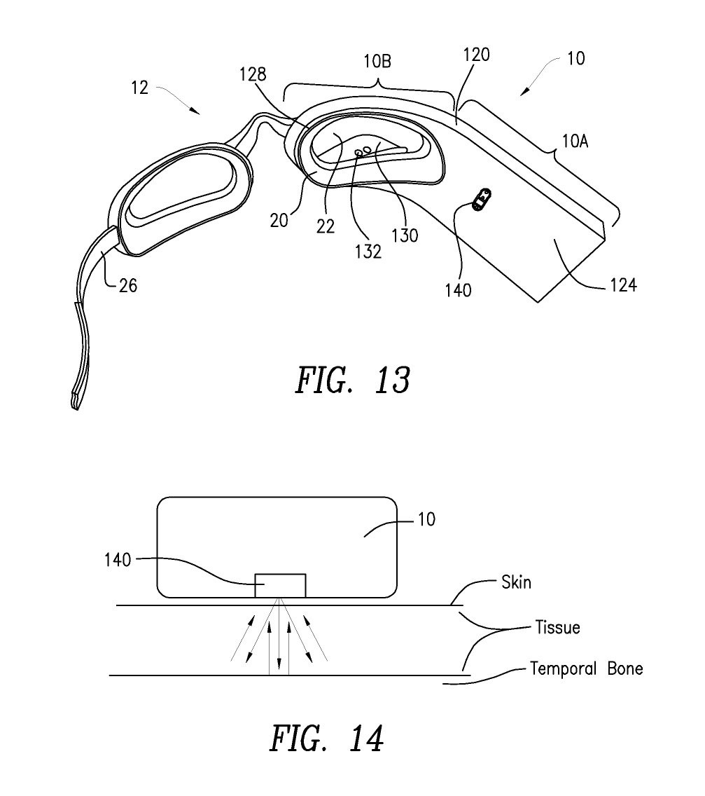

In use, the device 10 may fit between, for example, the strap 26 of a pair of swimming goggles 12 (or an arm of a pair of sunglasses) and the user's temple. As shown in FIGS. 12 and 13, a first portion 10A of the device may be adjacent to the user's right temple and a second portion 10B of the device may be curved in front of the user's right eye toward the user's nose; however, it will be understood that the device 10 may be alternatively configured to fit adjacent to the user's left temple and left eye. Although the shape of the housing unit 120 may smoothly transition between the first portion 10A and the second portion 10B in a smooth curve, at least a portion the first portion 10A may lie in a first plane and at least a portion of the second portion 10B may lie in a plane, and the first plane may be at least substantially orthogonal to the second plane. The strap 26 may pass between the strap attachment element 126 and the body of the housing unit 120. The strap attachment element 126 may fit tightly over the strap 26, thereby helping to keep the device 10 in place. The opening 128 of the housing unit 120 may be disposed about one of the eye cups 20 of the goggles 12, with at least a portion of the LED housing portion 130 being located anterior to a lower edge of the eye cup when worn by the user. That is, the lens 22 of the eye cup 20 may be disposed between the user's eye and the one or more LEDs 132 within the LED housing portion 130. In this manner, light emitted by the one or more LEDs 132 may cast light on the lens 22 of the eye cup 20 so that the light is indirectly viewable by the user. As the housing unit 120 may be composed of a soft, flexible material such as PTE, the relatively flexible portion of the housing unit 120 surrounding the opening 128 may be easily stretched over any of a variety of eyewear component shapes and sizes (for example, an eye cup of a pair of goggles or the frame surrounding a lens of a pair of sunglasses), thereby making the device 10 usable with the user's favorite eyewear.

The second surface 124 of the housing 130 may include an opening 138 for a heart rate measurement apparatus 140. The opening 138 may include a gasket or similar element for preventing water and/or other environmental contaminants from entering the housing unit 120. Further, the device 10 may optionally include a thin layer of transparent material within the housing unit 120 between the opening 138 and the heart rate measurement apparatus 140, which may offer further protection from water and/or other contaminants. For example, clear material such as epoxy may be inserted, molded, or otherwise present in the opening 138, so that the housing unit 120 is waterproof but still allows light to pass through. Any material may be used for this purpose, as long as its reflection properties are the same as or approximate glass.

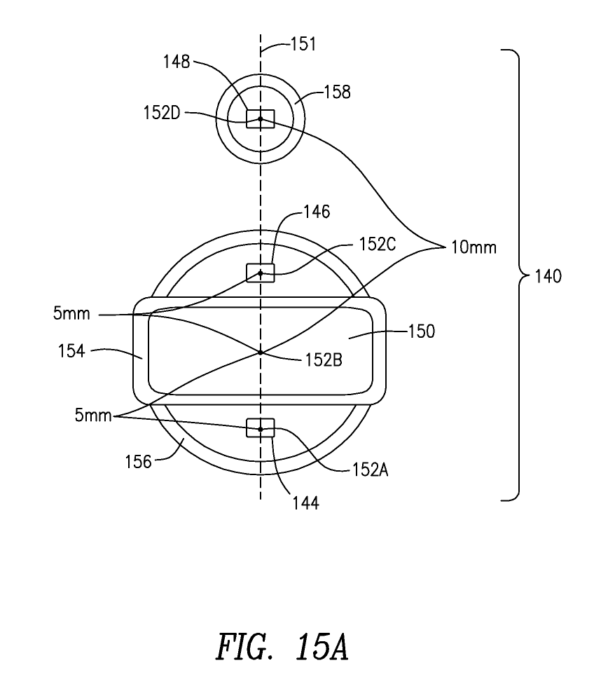

Instead of the infrared emitter 74 (photodiode) and infrared receiver 76 (photodiode) used in the heart rate measurement apparatus 30 in FIGS. 1-9, the heart rate measurement apparatus 140 of FIGS. 10-17 may include a plurality of light emission elements, such as LEDs. For example, the heart rate measurement apparatus 140 may include a first LED emitter 144, a second LED emitter 146, a third LED emitter 148, and a sensor 150. Each of the LED emitters 144, 146, 148 may emit green light rather than infrared light, and the sensor 150 may be configured to sense or receive green light that has been emitted toward the target reflection point 84 and reflected back toward the sensor 150. As a non-limiting embodiment, each LED emitter may emit green light having a wavelength between approximately 515 nm and approximately 525 nm. In use, light may be emitted from one or more of the LED emitters 144, 146, 148 through the user's skin and tissue (including capillaries) of the user's temple toward the temporal bone (as shown in FIG. 14). Although the temporal bone may be a preferred target, some light may reflect back to the sensor 150 from tissue between the temporal bone and the skin of the user's temple. So, in this respect, this tissue may also be referred to as a target reflection point, as long as the reflected light is providing heart rate signals to the sensor 150. Light then reflects from the temporal bone back through blood and tissue proximate the temporal bone, toward the sensor 150. In this embodiment, the reflection point 84 may be one or more locations on the temporal bone. The volume of blood in the capillaries varies with the user's heart beat and, in general, the higher the volume of blood within the capillaries in the user's temple, the less light that will be reflected from the temporal bone back to the sensor 150. Experiments showed that a better heart rate signal (that is, a higher amount of reflectance and/or more light transmission through temporal tissue) was produced using green light than any other wavelengths of light, at least when using the heart rate measurement apparatus 140 shown and descried in FIGS. 10-17.

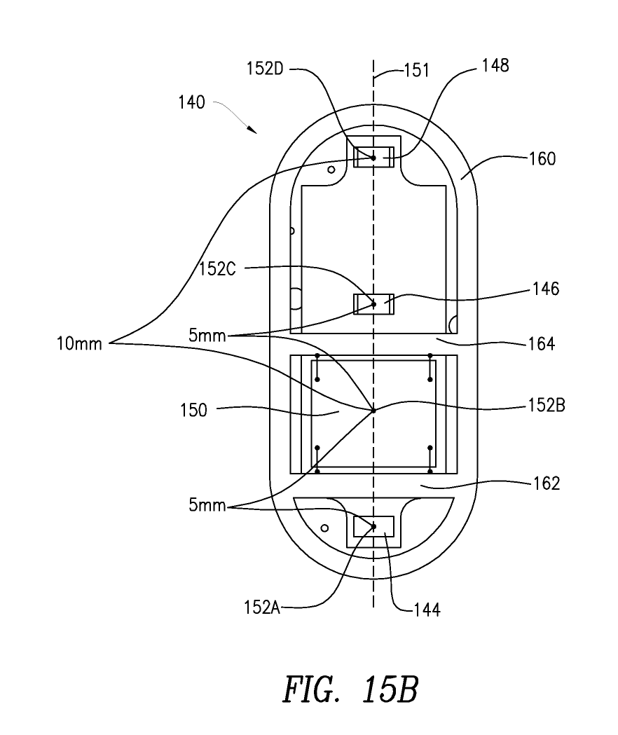

The sensor 150 generally may be larger than the three LED emitters 144, 146, 148, and may be located between the first 144 and second 146 LED emitters. For example, the first 144 and second 146 LED emitters may each be approximately 5 mm from the center point 152 of the sensor (depicted with an imaginary dot in FIGS. 15A and 15B), and each of the first 144 and second 146 LED emitters may be located opposite each other, on either side of the sensor 150. As shown in both FIGS. 15A and 15B, the center point 152A of the first LED emitter 144 may be located approximately 5 mm from the center point 152B of the sensor 150 in a first direction, and the center point 152C of the second LED emitter 146 may be located approximately 5 mm from the center point 152B of the sensor 150 in a second direction that is approximately 180° from the first direction. The center point 152D of the third LED emitter 148 may be located approximately 10 mm from the center point 152B of the sensor 150 in the second direction. All three LED emitters 144, 146, 148 may lie in a common imaginary line 151, depicted in dashed lines in FIGS. 15A and 15B. This configuration of LED emitters 144, 146, 148 and sensor 150 may make it possible to detect a good heart rate signal despite the thickness of tissues, distribution of capillaries in the temporal area, blood volume between the skin and temporal bone, skin color, and hair color, etc., all of which may vary widely between users.

In the embodiment shown in FIG. 15A, the sensor 150 may be substantially rectangular and may be surrounded by a sensor frame 154 that may be in contact with all four edges of the sensor 150 or may be slightly larger than the sensor. Additionally, the sensor frame 154 may be part of a substantially circular main frame 156 that surrounds the sensor 150, the first LED emitter 144, and the second LED emitter 146. Further, the third LED emitter 148 may be surrounded by a third LED emitter frame 158 that is separate and a distance from the main frame 156. Each of the sensor frame 154, the main frame 156, and the third LED emitter frame 158 may be raised from the surface of the housing unit 120 and the sensor 150 and LED emitters 144, 146, 148, functioning as partitions or shields between the sensor 150 and LED emitters 144, 146, 148 and between the light emitters 144, 146, 148 themselves. The raised frames 154, 156, 158 may help prevent light from the LED emitters 144, 146, 148 from being directly received by the sensor 150 before it can be reflected from the target reflection point 84.

In the embodiment shown in FIG. 15B, the sensor 150 may be substantially square, but the configuration of the LED emitters 144, 146, 148 may be the same as in the heart rate measurement apparatus configuration shown in FIG. 15A. That is, each of the first 144 and second 146 LED emitters may be located approximately 5 mm from the center point 152 of the sensor 150 in opposite directions, and the third LED emitter 148 may be located approximately 10 mm from the center point 152 of the sensor 150 in the same direction as the second LED emitter 146. In the embodiment of FIG. 15B, however, a single frame 160 may be used, and the frame 160 may include a first partition 162 between the first LED emitter 144 and the sensor 150 and a second partition 164 between the sensor 150 and the third LED emitter 148. Like the frames in FIG. 15A, the frame 160 in FIG. 15B may prevent light from the LED emitters 144, 146, 148 from being directly received by the sensor 150 before the light can be reflected from the target reflection point 84. Although two configurations of the heart rate measurement apparatus 140 are shown in FIGS. 15A and 15B, it will be understood that the sensor 150, LED emitters 144, 146, 148, and any frames used (for example, frames 154, 156, 158, and 160) may have any suitable size and shape, and the frames may have any suitable configuration.

In addition to a microcontroller 34, the device 10 may also include components within the housing unit 120, such as a power source 39, a BLUETOOTH® chip and/or ANT™ chip 165, a three-axis gyroscope 166, a three-axis accelerometer 168, a three-axis magnetometer 170, an ambient light sensor 172, a USB connector 174, and one or more user input devices, such as one or more buttons 176 for starting and stopping biofeedback recording, entering the device into a sleep mode, powering the device on/off, and/or other functions. The ambient light sensor 172 may detect the intensity of environmental or ambient light and may adjust the brightness of the one or more LEDs 132 and/or LED emitters 144, 146, 148 accordingly. For example, if the ambient light is bright, the ambient light sensor 172 may increase the brightness of the one or more LEDs 132 and/or LED emitters 144, 146, 148 to compensate. The power source 39 may be rechargeable. For example, the power source 39 may recharge when the device 10 is connected via the USB connector 174 to a computer or wall outlet. As shown in FIG. 12, the USB connector 174 may be selectively concealed or exposed using a USB cover portion 178 of the housing unit 120. For example, the USB cover portion 178 may be foldable about the USB connector 174 and may include one or more matable tabs 180 and indentations 182 that provide a waterproof seal about the USB connector 174 when the USB connector 174 is concealed, but that are also easily disengageable when the user desires to expose the USB connector 174 to connect to a computer or wall outlet (for example, as shown in FIG. 11). In alternative embodiments, the USB cover portion 178 may be a removable cap that is composed of the same material as the housing unit 120 or another waterproof material (such as plastic), or the USB cover portion 178 may be a pocket that is integrated with the housing unit 120, and composed of the same flexible material as the housing unit 120, that can be stretched over the USB connector 174. The USB connector 174 may be in direct or indirect electrical communication with the microcontroller 34, the three-axis gyroscope 166, the accelerometer 168, the magnetometer 170, the ambient light sensor 172, the one or more LEDs 132, the sensor 150, and the LED emitters 144, 146, 148.

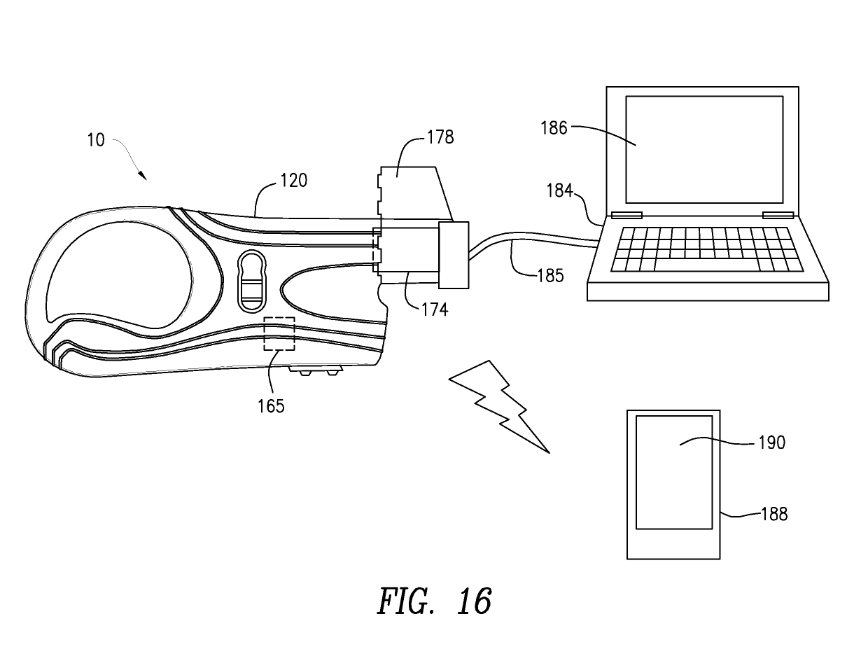

As shown in FIG. 16, the device 10 may communicate with computer or mobile device software via the USB connector 174 and/or BLUETOOTH® chip and/or ANT™ chip 165. For example, the USB connector 174 may be plugged directly into a computer 184 or via an extension or adapter cable 185. As a non-limiting embodiment, connecting the device 10 to a computer 184 may initiate a program display 186. The program software may reside locally on the computer's hard drive, within cloud storage, or both. The program may include the display of a “dashboard” or user data interface. Additionally or alternatively, the device 10 may communicate to a mobile device 188 running application software featuring a display 190. For example, the program may display to the user a graphical representation of the user's heart beat as detected by the sensor 150, the number of laps the user swam and number of turns the user made (measured, for example, by the three-axis gyroscope 166), the number of calories burned (measured, for example, by the sensor 150 and the accelerometer 168), and total distance and instantaneous and/or average speed (measured, for example, by the accelerometer 168, three-axis gyroscope 166, and/or magnetometer 170). Although this recorded data may be saved to a memory chip within the device 10 and displayed on the software dashboard when the user connects the device 10 to a computer 184 or mobile device 188, the data may also be displayed on a small screen in the LED housing portion and/or communicated to the user via, for example, the LED lights 132 or audio components within the device. For example, the device 10 may include a small speaker 192 within the housing unit 120 and configured to produce a volume that is audible by the user through the housing unit 120. Alternatively, the housing may include waterproof perforations or an opening in the second side 124 of the housing unit 120 through which sound emitted by the speaker 192 may pass. The speaker 192 may communicate data to the user in a human or humanlike voice in a language understandable by the user (language preferences may be set using the software). Additionally or alternatively, the speaker 192 may communicate data to the user by one or more audio tones. The device 10 may also include a vibration mechanism, and certain data and/or training guidance may be similarly communicated to the user by haptic feedback. For example, the device 10 may include a small motor 194 that may vibrate against the user's temple when the device registers a change in direction (such as a flip turn), when the user's heart rate passes a target level, or when the user's current lap time is faster than the last lap.

The user may also enter data such as age, height, weight, and/or body type into the software, which may then be communicated to the device 10. Optionally, the user may enter one or more planned workouts into the software, which may be communicated to the device 10. As a non-limiting example, the software may compare the user's entered data (height, weight, age, etc.) with a planned workout and/or one or more manually entered and/or automatically generated parameters, such as target heart rate, target time within a particular heart rate zone, target calories burned, target workout duration, target distance, target number of laps, etc. During an activity, the device 10 may use this data to generate one or more audible or visual alerts to the user indicating target criteria have been reached and/or that the user needs to adjust activity to, for example, raise or lower heart rate to meet target criteria.

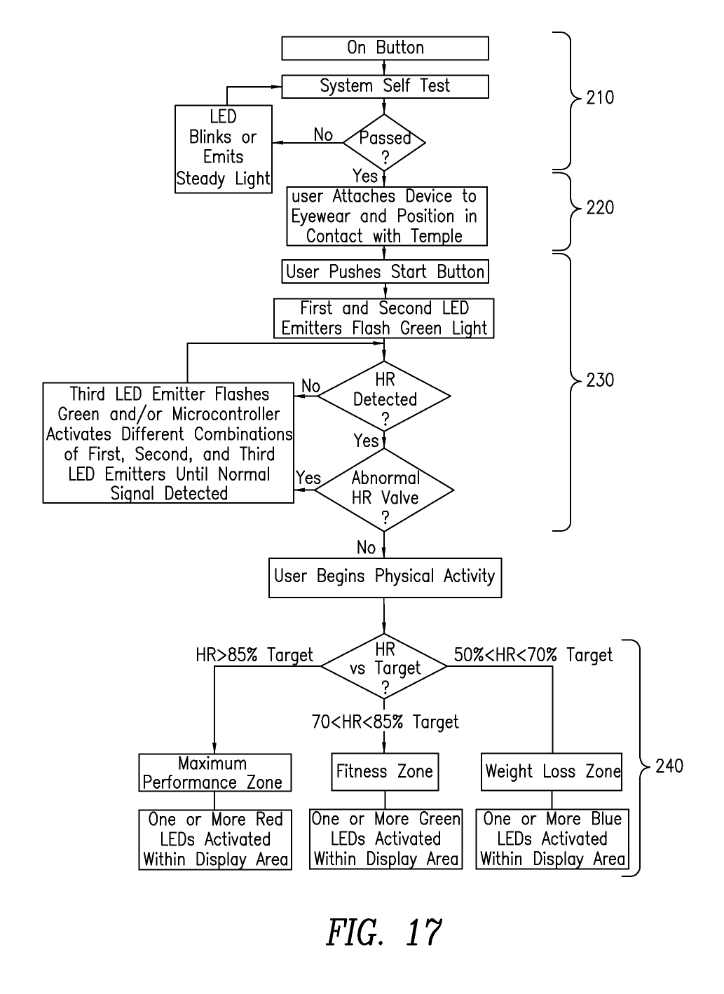

A non-limiting exemplary flowchart of device 10 operation is shown in FIG. 17. Similar to the heart rate measurement apparatus 30 of FIGS. 1-9, the heart rate measurement apparatus 140 of FIGS. 10-17 may be in electrical communication with a microcontroller 34 that may receive and process heart rate and other biofeedback signals from the sensor 150. When the device 10 is turned on, the device 10 may undergo a self test, as shown and described in FIG. 9. For example, in a first step 210 the user may turn on the device (such as by pressing one of the buttons 176) and the device 10 may verify that all components are functioning properly and are in communication with each other and/or the microcontroller 34. One or more of the LEDs 132 may blink or steadily emit light to indicate to the user that the device 10 either passed or failed the self test. If the self test fails, the user may power cycle the device 10 or perform another reset function. In a second step 220 the user may attach the device 10 to an item of eyewear, such as a pair of swimming goggles 12 as shown in FIGS. 15A and 15B. Then, the user may position the device 10 such that the heart rate measuring apparatus 140 is in contact with his or her temple. However, it will be understood that the user may first attach the device to an item of eyewear and then turn on the device.

In the third step 230, the user may push a start button 176. The first 144 and second 146 LED emitters may become activated when the device is started, and they may emit light in discrete bursts (that is, the LEDs 144, 146 may flash rather than emit continuous light) toward the temporal bone for a startup period of up to approximately 10 seconds. If the heart rate measurement apparatus 140 (for example, the sensor 150) is unable to detect a good heart rate signal within this startup period, the microcontroller 34 may be programmed to activate the third LED emitter 148, which may also emit light in discrete bursts toward the temporal bone until a good heart rate signal is detected. The microcontroller 34 may also be programmed to activate or deactivate one or more LED emitters to find a green light emission from one or more LED emitters that sufficiently reflects from the target reflection point 84 and is received by the sensor 150. For example, if no heart rate signal or an abnormal heart rate signal is detected by the sensor 150, the microcontroller may activate different combinations of the first 144, second 146, and third 148 LED emitters until a normal heart rate signal is detected. It will be understood that the term “target reflection point” may be used to refer to any reflection point within a user's temple that emits sufficient light to the sensor 150 for the sensor 150 to detect a good heart rate or other biofeedback signal, and may not be in a specific location but rather a location that varies between users. Additionally, the target reflection point 84 may be a plurality of locations. For example, green light may be emitted generally toward the user's temporal bone, and the light may be reflected back to the sensor 150 from one or more locations on the temporal bone and/or tissue between the temporal bone and the skin of the user's temple.

In the fourth step 240, the user may begin his or her physical activity. In a non-limiting example, the display area 134 may show light transmitted from one or more blue LEDs to indicate the user is operating at a heart rate within a fat-burning zone, may show light transmitted from one or more green LEDs to indicate the user is operating at a heart rate within a fitness zone, and one or more red LEDs to indicate the user is operating at a heart rate within a maximum performance zone. Optionally, the one or more LEDs may also blink quickly or slowly to indicate particular ranges within a zone, as shown and described in FIG. 9. Additionally, the display may show light from an orange LED to indicate the current battery level. For example, the orange LED may flash when the battery level is below 25%.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed:

1. A heart rate measurement apparatus, the apparatus comprising:
a first light emission element, a second light emission element, and a third light emission element, each of the first, second, and third light emission elements being configured to emit green light toward a target location; and
a light sensor configured to receive green light reflected from the target location, the sensor being located between the first light emission element and the second light emission element, the first light emission element, sensor, second light emission element, and third light emission element being at least substantially collinear.

2. The apparatus of claim 1, wherein the first light emission element is located approximately 5 mm from the sensor in a first direction, the second light emission element is located approximately 1 cm from the sensor in a second direction, and the third light emission element is located approximately 10 mm from the sensor in the second direction.

3. The apparatus of claim 2, wherein the first direction is approximately 180° from the second direction.

4. The apparatus of claim 3, further comprising a first shield element between the first light emission element and the sensor, a second shield element between the sensor and the second light emission element, and at least one shield element between the second light emission element and the third light emission element.

5. The apparatus of claim 3, wherein each of the first, second, and third light emission elements are light-emitting diodes.

6. The apparatus of claim 5, wherein the apparatus is located within a housing.

7. The apparatus of claim 6, wherein the housing has a first portion and a second portion, each of the first portion and the second portion having a first side and a second side.

8. The apparatus of claim 7, wherein at least a portion of the housing first portion lies in a first plane and at least a portion of the housing second portion lies in a second plane, the first plane being at least substantially orthogonal to the second plane.

9. The apparatus of claim 8, wherein the second side of the housing first portion includes an opening sized to allow at least a portion of the heart rate measurement apparatus to extend therethrough.

10. The apparatus of claim 9, wherein the first light emission element, the second emission element, and the third emission element emit light through the opening in the second side of the housing first portion, and the sensor detects light reflected from the reflection location through the opening in the second side of the housing first portion.

11. The apparatus of claim 10, wherein the housing second portion defines an opening that extends from the second portion first side to the second portion second side.

12. The apparatus of claim 11, the housing including a plurality of signal light emission elements disposed within the housing second portion, the housing being configured such that light emitted from the plurality of signal light emission elements is visible through the second side of the housing second portion.

13. The apparatus of claim 12, wherein the housing is configured to be releasably engageable with an item of eyewear.

14. The apparatus of claim 13, wherein the item of eyewear is a pair of swimming goggles having an eye cup, the eye cup defining an outer perimeter and including a lens having an anterior face.

15. The apparatus of claim 14, wherein the housing second portion opening is configured to be disposed about the outer perimeter of the eye cup, and the plurality of light emission elements configured to emit light onto the anterior face of the eye cup lens.

16. The apparatus of claim 1, wherein the target location is a temporal bone.

17. A biofeedback device, the device comprising:
a housing defining a first portion and a second portion, each of the first portion and second portion defining a first side and a second side, the housing further defining a first opening in the second side of the housing first portion and a second opening that extends from the first side of the housing second portion to the second side of the housing second portion;
a plurality of signal light emission elements disposed within the housing second portion and being configured to emit light through the housing second portion; and
a heart rate measurement apparatus at least partially disposed within the housing, the heart rate measurement apparatus including:
a first light emission element, a second light emission element, and a third light emission element, each of the first, second, and third light emission elements being configured to emit light toward a target location; and
a light sensor configured to receive light reflected from the target location, the sensor being located between the first light emission element and the second light emission element, the first light emission element, sensor, second light emission element, and third light emission element being at least substantially collinear and positioned within the first opening.

18. The biofeedback device of claim 17, wherein the first light emission element, the second light emission element, and the third light emission element are configured to emit green light and the sensor is configured to detect green light reflected from the target location.

19. The biofeedback device of claim 18, wherein at least a portion of the housing first portion lies in a first plane and at least a portion of the housing second portion lies in a second plane, the first plane being substantially orthogonal to the second plane.

20. The biofeedback device of claim 19, wherein the target location is within a carotid artery.

21. A biofeedback system, the system comprising:
a pair of swimming goggles including an eye cup defining an outer perimeter and including a lens defining an anterior face;
a housing defining a first portion and a second portion, each of the first portion and second portion defining a first side and a second side, the housing further defining a first opening in the second side of the housing first portion and a second opening that extends from the first side of the housing second portion to the second side of the housing second portion;
a plurality of signal light emission elements disposed within the housing second portion and being configured to emit light through the housing second portion; and
a heart rate measurement apparatus at least partially disposed within the housing, the heart rate measurement apparatus including:
a first light emission element, a second light emission element, and a third light emission element, each of the first, second, and third light emission elements being configured to emit green light toward a target location; and
a light sensor configured to receive green light reflected from the target location, the sensor being located between the first light emission element and the second light emission element, the first light emission element, sensor, second light emission element, and third light emission element being at least substantially collinear and positioned within the first opening.
